(19) United States
(12) Patent Application Publication
(10) Pub. No.: US 2022/0260472 A1
(43) Pub. Date: Aug. 18, 2022

(54) EVALUATION METHOD OF CULTURE SUBSTRATE AND/OR CULTURE MEDIUM SOLUTION, AND USE OF EVALUATION METHOD

(71) Applicant: SUMIKA CHEMICAL ANALYSIS SERVICE, LTD., Osaka (JP)

(72) Inventors: Yosuke AZUMA, Osaka (JP); Orie TERAI, Osaka (JP); Miki IWATA, Osaka (JP); Katsuya IMANISHI, Osaka (JP)

(21) Appl. No.: 17/619,517

(22) PCT Filed: Jun. 15, 2020
(86) PCT No.: PCT/JP2020/023334

§ 371 Date: Dec. 15, 2021

(30) Foreign Application Priority Data
Jun. 17, 2019 (JP) 2019-112204

(51) Int. Cl. G01N 13/02; C12N 5/0775

(52) U.S. Cl. CPC G01N 13/02; G01N 2013/0208; C12N 5/0662

(57) ABSTRACT

The present invention provides a method for simply evaluating whether or not a culture substrate and/or a culture medium solution used in cell culture is suitable for culturing intended cells. The evaluation method in accordance with the present invention measures an affinity (e.g., contact angle) between a culture substrate and a culture medium solution, and evaluates whether or not the culture substrate and/or the culture medium solution is suitable for culturing intended cells based on the result of measuring the affinity between the culture substrate and the culture medium solution.

### TECHNICAL FIELD

The present invention relates to a method for evaluating suitability of a culture substrate and/or a culture medium solution used in various kinds of cell culture. Specifically, the present invention relates to an evaluation method which makes it possible to simply evaluate whether or not a culture substrate and/or a culture medium solution used in cell culture is suitable for culturing intended cells.

In addition, the present invention relates to utilization examples (a cell culture method, a method for searching a culture substrate and/or a culture medium solution used in cell culture) of the above evaluation method.

### BACKGROUND ART

In production of cells for use in the field of regenerative medical technique and in production of substances for pharmaceuticals using cells and for compounds used in other applications, efficient and stable production of an appropriate amount of good quality cells is an important objective. In general, it is known that properties of cultured cells and culture efficiencies vary greatly in accordance with a culture substrate and a culture medium solution used in cell culture. Under the circumstances, in order to efficiently culture cells which stably have intended properties, development of a culture substrate and a culture medium solution which are suitable for the cell culture is being carried out. However, depending on cells, appropriate combinations of a culture substrate and a culture medium solution are different. Therefore, at present, it is necessary that various culture substrates and various culture medium solutions are combined and cells are actually cultured, in order to evaluate which culture substrate and culture medium solution are suitable for culturing intended cells. Thus, much time and labor have been needed to select and develop a culture substrate and a culture medium solution.

As a method for evaluating a culture substrate, for example, a method disclosed in Patent Literature 1 is known. The method for evaluating a culture substrate disclosed in Patent Literature 1 is a method for evaluating a cell culture substrate including a substrate and a functional layer which is provided on the substrate for culturing cells. The method includes: a step of supplying a droplet onto a surface of the functional layer; a step of further introducing liquid into the supplied droplet to expand the droplet, or a step of sucking liquid from the supplied droplet to contract the droplet; and a step of measuring a contact angle of the droplet to be expanded or contracted. In this method, phenomena in which the contact angle repeatedly increases and decreases are observed, and a state of the functional layer is evaluated based on the observation result. According to the method for evaluating a culture substrate disclosed in Patent Literature 1, it is possible to evaluate the state (quality of desorption of the cells) of the functional layer which is made of poly-N-isopropylacrylamide (PIPAAm) or the like and is provided on the culture substrate.

### CITATION LIST

### Patent Literature

[Patent Literature 1]

Japanese Patent Application Publication Tokukai No. 2013-192544 (Publication date: Sep. 30, 2013)

### SUMMARY OF INVENTION

### Technical Problem

However, the evaluation method disclosed in Patent Literature 1 is merely a method for evaluating the quality of desorption of a cell sheet which has been cultured on the surface of the functional layer made of PIPAAm or the like. Therefore, the evaluation method disclosed in Patent Literature 1 cannot evaluate whether or not a culture substrate and/or a culture medium solution used in cell culture is suitable for culturing intended cells.

In view of this, an object of the present invention is to provide a method for simply evaluating whether or not a culture substrate and/or a culture medium solution used in cell culture is suitable for culturing intended cells. In addition, the present invention provides utilization examples (a cell culture method, a method for searching a culture substrate and/or a culture medium solution used in cell culture) of the evaluation method.

### Solution to Problem

In order to attain the object, the inventors of the present invention have diligently studied and, as a result, the present invention has been accomplished by finding that an affinity between a culture substrate and a culture medium solution used in culturing (e.g., a contact angle of a droplet of the culture medium solution supplied on the culture substrate) is related to an outcome of cell culture (such as properties of cells, growth rate, culture efficiency, area of adhesion to the culture substrate).

That is, an evaluation method in accordance with an embodiment of the present invention is a method for evaluating suitability of a culture substrate and/or a culture medium solution used in cell culture, the method including: an affinity measuring step of measuring an affinity between the culture substrate and the culture medium solution; and a suitability determining step of determining suitability of the culture substrate and/or the culture medium solution based on a result of measuring the affinity between the culture substrate and the culture medium solution which has been obtained in the affinity measuring step.

In the evaluation method in accordance with an embodiment of the present invention, it is possible that the suitability determining step is carried out based on a suitable criterion of affinity which has been determined in advance.

In the evaluation method in accordance with an embodiment of the present invention, it is possible that, in the affinity measuring step, a contact angle between the culture substrate and a droplet of the culture medium solution is measured.

It is possible that the evaluation method in accordance with an embodiment of the present invention further includes: a criterion determining step of determining a suitable criterion of affinity between the culture substrate and the culture medium solution in advance.

The present invention can be a method for culturing cells, the method including the step of evaluating, by the foregoing evaluation method, suitability of a culture substrate and/or a culture medium solution used in cell culture.

The present invention can be a method for searching a culture substrate and/or a culture medium solution used in cell culture, the method including the step of evaluating, by the foregoing evaluation method, suitability of a culture substrate and/or a culture medium solution used in cell culture.

Note that, in the evaluation method disclosed in Patent Literature 1, an affinity of the functional layer on the culture substrate with respect to water is evaluated, and therefore a droplet of water or the like is supplied in a form of a functional layer to measure the contact angle. In contrast, in the evaluation method in accordance with an aspect of the present invention, an affinity between a culture medium solution and a culture substrate is evaluated by measuring the contact angle or the like. In this respect, the two inventions are clearly different.

### Advantageous Effects of Invention

According to the present invention, it is possible to simply evaluate whether or not a culture substrate and/or a culture medium solution used in cell culture is suitable for culturing intended cells.

### BRIEF DESCRIPTION OF DRAWINGS

FIG. 1 is a schematic view for explaining a contact angle.

FIG. 2 is a microscopic image of a droplet 60 seconds after pure water or a culture medium solution is dripped and fixed on a surface of each of various commercially available culture substrates (dishes). (a) of FIG. 2 shows a microscopic image of a case where pure water was dripped onto a commercially available culture substrate (referred to as “dish A”). (b) of FIG. 2 shows a microscopic image of a case where pure water was dripped onto another commercially available culture substrate (referred to as “dish B”). (c) of FIG. 2 shows a microscopic image of a case where pure water was dripped onto another commercially available culture substrate (referred to as “dish C”). (d) of FIG. 2 shows a microscopic image of a case where a culture medium solution was dripped onto the dish A. (e) of FIG. 2 shows a microscopic image of a case where a culture medium solution was dripped onto the dish B. (f) of FIG. 2 shows a microscopic image of a case where a culture medium solution was dripped onto the dish C.

FIG. 3 shows microscopic images of human mesenchymal stem cells cultured on various culture substrates (dishes), the images being taken immediately after seeding (after 4 hours of culture) and at a time of reaching confluence (after 11 days of culture).

FIG. 4 is graphs showing the following properties of human mesenchymal stem cells cultured for 4 hours on various culture substrates (dishes): (a) the number of cells adhered to the culture substrate (the number of adherent cells); (b) a total adhesion area of cells made contact with the culture substrate; and (c) adhesion area/number of adherent cells.

FIG. 5 is a line graph showing change of the number of cells with time when human mesenchymal stem cells are cultured on various culture substrates (dishes). The number of cells is expressed as a relative percentage (relative number of cells) at each culture time (i.e., 4 hours later, 24 hours later, 72 hours later), where the number of cells immediately after seeding (i.e., after 4 hours of culture) is assumed to be 100%.

### DESCRIPTION OF EMBODIMENTS

The description below deals with an embodiment of the present invention in detail. Note, however, that the present invention is not limited to the embodiment, and can be made in an aspect obtained by variously altering the embodiment within the described scope. Moreover, all scientific literatures and patent literatures described in this specification are incorporated herein as reference. Note that a numerical range “A to B” herein means “A or more (higher) and B or less (lower)” unless otherwise stated.

An embodiment of the present invention is a method for evaluating suitability of a culture substrate and/or a culture medium solution used in cell culture, the method including: an affinity measuring step of measuring an affinity between the culture substrate and the culture medium solution; and a suitability determining step of determining suitability of the culture substrate based on a result of measuring the affinity between the culture substrate and the culture medium solution which has been obtained in the affinity measuring step (hereinafter referred to as “present evaluation method” as appropriate).

The phrase “evaluating suitability of a culture substrate and/or a culture medium solution used in cell culture” means to evaluate, when intended cells are cultured using a certain culture substrate and/or a certain culture medium solution (hereinafter referred to as “culture substrate, and/or the like” as appropriate), whether or not the culture substrate and/or the like is suitable for culturing the intended cells in terms of properties (such as morphology and activity) of cells, growth rate, culturing efficiency, adhesion area to the culture substrate, and the like.

(1) Affinity Measuring Step

The affinity measuring step in the present evaluation method is a step of measuring an affinity between a culture substrate which is used in cell culture and a culture medium solution which is used in the cell culture.

The term “cell culture” in the present evaluation method means not only culture of animal cells but also culture of plant cells, insect cells, and microorganisms such as bacteria and yeasts. In this specification, culture of animal cells is described as a typical example of cell culture but the present invention is not limited to this example. Note that the method for culturing animal cells can be an adhesive culture method or a suspension culture method.

Examples of the animal cells include, but are not particularly limited to, cartilage cells, osteoblasts, odontoblasts, ameloblasts, mammary epithelial cells, ciliated epithelium cells, intestinal epithelium cells, fat cells, hepatocytes, mesangial cells, glomerular epithelial cells, sinusoidal endothelial cells, Kupffer cells, myoblasts, nerve cells, glial cells, fibroblasts, smooth muscle cells, stem cells (such as ES cells, interstitial cells, mesenchymal stem cells, neural stem cells), progenitor cells thereof, and the like. Examples of the origin of the animal cells include, but are not particularly limited to, human, monkey, dog, cat, rabbit, rat, nude mouse, mouse, guinea pig, pig, sheep, Chinese hamster, cattle, marmoset, African green monkey, and the like.

Although the animal cells are not particularly limited, it is preferable that the animal cells are established cells because culture can be stably carried out. Examples of such a cell line include, but are not particularly limited to, NIH/3T3 cell line (mouse fetal fibroblasts), 3T3-Swiss albino cell line (mouse fetal fibroblasts), A549 cell line (human pulmonary adenocarcinoma cells), HeLa cell line (human cervical epidermoid tumor cells), Vero cell line (African green monkey normal kidney cells), 293 (human fetal kidney cells), 3T3-L1 (mouse fibroblasts), HepG2 (human liver cancer-derived cells), MCF-7 (human breast cancer-derived cells), V79 (Chinese hamster-derived fibroblasts), COS-7 (African green monkey kidney-derived cells), CHO-K1 (Chinese hamster ovary-derived cells), WI-38 (human lung fibroblasts), MDCK (canine kidney-derived cells), MRC-5 (normal lung fibroblasts), bovine vascular endothelial cells, and the like. In Examples described later, cell culture was carried out using human mesenchymal stem cells (PT-2501, Lonza Japan Ltd.)

The animal cells can be artificially produced cells such as induced pluripotent stem (iPS) cells. Differentiated cells derived from iPS cells are cells used for living organ transplantation. Therefore, efficient preparation of a cell sheet for living organ transplantation using iPS cells is extremely beneficial in the field of regenerative therapy.

In the present invention, the “culture substrate used in cell culture” means a portion of a culture vessel (such as petri dish, flask, plate, culture bag, microbeads, microfibers) used in cell culture in which a culture medium and cells are in direct contact during a culture period. For example, the term means an inner wall surface of a petri dish or the like. In cell culture, it is known that physical properties or the like of a culture substrate, which serves as a scaffold for cells, affect growth and differentiation of cells. Therefore, it can be said that significance of evaluating the culture substrate in the present invention is large.

A material constituting a culture substrate in the present invention is not particularly limited as long as the material can be used in cell culture. For example, a synthetic resin, silicone, glass, or the like can be used as a material constituting a culture substrate. From the viewpoint of cost and visibility of cells during microscope observation, it is preferable to use a transparent synthetic resin as the material. Examples of the transparent synthetic resin include: acrylic resins such as polymethyl methacrylate and a methyl methacrylate-styrene copolymer; styrene-based resins such as polystyrene; olefin-based resins such as cycloolefin; ester-based resins such as polyethylene terephthalate and polylactic acid; silicone-based resins such as polydimethylsiloxane; polycarbonate resins; and the like. Such resins can contain various additives such as a colorant, a diffusing agent, a thickener, and the like within a range not impairing transparency.

A surface of a culture substrate in the present invention can be subjected to various surface treatments or can be provided with various coating layers constituted by materials such as laminin, collagen, and polylysine in order to improve hydrophilicity, biocompatibility, cellular affinity, and the like of the surface. A functional layer made of poly-N-isopropylacrylamide (PIPAAm) or the like can be provided on the culture substrate.

The surface treatment is not particularly limited, and examples of the surface treatment include: chemical treatments such as chemical agent treatment, solvent treatment, and graft polymer introduction by surface graft polymerization; and physical treatments such as corona discharge, ozone treatment, and plasma treatment. The method of providing the coating layer is not particularly limited, and examples of the method include: dry coating such as sputtering and vapor deposition; wet coating such as inorganic material coating and polymer coating; and the like.

A “culture medium solution” used in the present invention can be a culture medium itself capable of carrying out culture of intended cells. Meanwhile, it is not necessary to include all components constituting the culture medium, and a solution can be employed which contains one or some of components such as a main component of the culture medium. As the culture medium, a known culture medium used in cell culture can be used as appropriate. Examples of the culture medium for culturing animal cells include Ham's F12 culture medium, α-MEM culture medium, DMEM culture medium, RPMI-1640 culture medium, MCDB201 culture medium, IMDM culture medium, and the like. These culture media can be used alone, or two or more types of these can be used as a mixture. Various additives such as serum, cell growth factors, antibiotic substances, amino acids, vitamins, and salts can be added to the culture medium.

The affinity measuring step in the present evaluation method is not particularly limited as long as the step is a process that can measure an affinity between a culture substrate and a culture medium solution. As the process that can measure an affinity between a culture substrate and a culture medium solution, the affinity can be measured by, for example, measuring a contact angle (also referred to as “wettability”) between a culture substrate and a droplet of a culture medium solution. Other methods include: a method in which a zeta potential, surface free energy, an SP value (Hildebrand solubility parameter), and an HSP value (Hansen solubility parameter) of a culture substrate and/or a culture medium solution are measured and evaluation values thereof are compared; a method for evaluating wettability disclosed in Japanese Patent Application Publication, Tokukai, No. 2019-20228; top surface observation of contact angle (method in which a wet state is photographed from above); and the like.

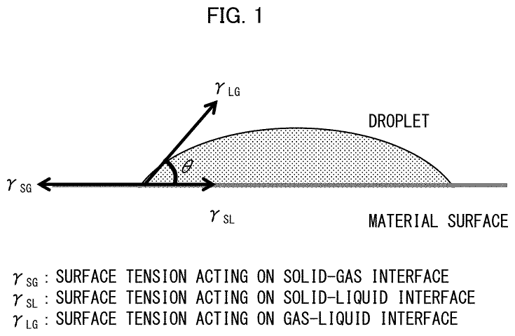

Among these methods, the method of measuring a contact angle between a culture substrate and a droplet of a culture medium solution is preferable from the viewpoint that an affinity between the culture substrate and the culture medium solution can be measured simply and quickly. As illustrated in FIG. 1, when a liquid is dripped onto a solid surface, the liquid is rounded due to its own surface tension and becomes a droplet. An angle θ between a tangent of the droplet and the solid surface is referred to as a “contact angle”. It can be said that a smaller contact angle indicates higher wettability of a liquid with respect to a solid substance (that is, an affinity between the liquid and the solid substance is high). In addition, it can be said a larger contact angle indicates lower wettability of a liquid with respect to a solid substance (that is, an affinity between the liquid and the solid substance is low).

A contact angle can be measured by a known method such as a θ/2 method, a tangential method, or a curve fitting method. Measurement of the contact angle can be carried out using a commercially available contact angle meter and in accordance with attached manuals. In Examples described later, the contact angle was measured using Drop Master 500 available from Kyowa Interface Science Co., Ltd. Note that the measurement method of the contact angle exemplified above is a method of measuring a contact angle in a condition in which a droplet of a culture medium solution is still on a culture substrate (method of measuring a static contact angle). However, the affinity measuring step in the present evaluation method can be carried out not only by the method of measuring a static contact angle but also by a method of measuring a contact angle in a condition in which a droplet of a culture medium solution is moving on a culture substrate (method of measuring a dynamic contact angle). The dynamic contact angle can be measured by, for example, a known method such as a sessile drop method, an expansion/contraction method, or a sliding method (falling method). The dynamic contact angle can also be measured using a commercially available measuring device and in accordance with attached manuals.

In any of the above measurement methods, there is no particular limitation on a temperature at the time of measurement and on atmosphere gas at the time of measurement as long as those matters fall within ranges in which the measurement can be accurately carried out. For example, the temperature at the time of measurement can be 0° C. to 10° C., 10° C. to 20° C., 20° C. to 25° C., 25° C. to 30° C., 30° C. to 40° C., or 40° C. to 50° C. Examples of the atmosphere gas at the time of measurement include air, nitrogen, oxygen, argon, krypton, CO₂, CO, water vapor, and the like. Note that two or more kinds of atmosphere gas can be mixed.

(2) Suitability Determining Step

The suitability determining step in the present evaluation method is a step of determining suitability of the culture substrate based on a result of measuring the affinity between the culture substrate and the culture medium solution which has been obtained in the affinity measuring step.

A greatest feature of the present evaluation method is that an affinity of a culture substrate with a culture medium solution containing a component of a culture medium used in cell culture is measured, instead of an affinity with water or the like as disclosed in Patent Literature 1. As indicated in Examples described later, a result of measuring a contact angle (affinity=hydrophilicity) between each of various culture substrates and a droplet of pure water did not conform to a result of measuring a contact angle (affinity) between each of various culture substrates and a droplet of a culture medium solution. Then, the inventors of the present invention uniquely found that there is a relationship between the result of measuring a contact angle (affinity) between each of various culture substrates and a droplet of a culture medium solution and a result of culture (morphology of cultured cells, area of adhesion of cultured cells to a culture substrate, relative number of cells obtained by culture). That is, according to this finding, it is possible to predict to some extent an outcome of cell culture by measuring an affinity between a culture substrate and a culture medium solution.

Note that the present invention provides a technical idea that an affinity between a culture substrate and a culture medium solution is related to an outcome of cell culture. In Examples described later, an outcome of cell culture was good when an affinity between a culture substrate and a culture medium solution was high. Moreover, when an affinity between a culture substrate and a culture medium solution is conversely low, an outcome of cell culture can be good.

Which one of the cases where (i) an affinity between a culture substrate and a culture medium solution is high and conversely (ii) an affinity between a culture substrate and a culture medium solution is low leads to the good outcome of cell culture can vary depending on a type of cells to be cultured, a type of a culture substrate, a type of a culture medium solution, and the like. Therefore, it is preferable to confirm, by experimentation in advance, which tendency would be shown.

In the evaluation method in accordance with an embodiment of the present invention, it is preferable to determine in advance a criterion of contact angle (i.e., a preferable range of contact angle or a criterion value of contact angle). As a method for determining the criterion of contact angle, first, culture is carried out while fixing a type of cells and a type of a culture medium solution and changing a type of a culture substrate. Next, a contact angle between the culture substrate and a droplet of the culture medium solution and an outcome of cell culture are analyzed to obtain the criterion of contact angle. Alternatively, culture can be carried out while fixing a type of cells and a type of a culture substrate and changing a type (or composition) of a culture medium solution. In this case also, a contact angle between the culture substrate and a droplet of the culture medium solution and an outcome of cell culture are analyzed to determine the criterion of contact angle (i.e., a preferable range of contact angle or a criterion value of contact angle). The evaluation method in accordance with an embodiment of the present invention can include a step (criterion determining step) of determining a criterion of affinity (i.e., a preferable range of contact angle or a criterion value of contact angle) between the culture substrate and the culture medium solution in advance.

As described above, the criterion of contact angle between a culture substrate and a droplet of a culture medium solution can be set in advance. From this, it is possible to simply evaluate whether or not a culture substrate and a culture medium solution are suitable for culturing intended cells only by measuring a contact angle between the culture substrate and a droplet of the culture medium solution without actually carrying out cell culture.

More specifically, for example, when a suitable culture substrate in culturing certain cells is searched, it is possible that a predetermined culture medium solution is supplied onto a culture substrate which is a search target, and a contact angle between the culture substrate and a droplet of the culture medium solution is measured. Then, whether or not the contact angle meets a predetermined criterion (whether or not the contact angle falls within a preferable range, or whether the contact angle exceeds or falls below the criterion value) is analyzed, and it is thus possible to determine whether or not the culture substrate is suitable for culturing the cells.

Alternatively, when a suitable culture medium solution in culturing certain cells is searched, it is possible that a culture medium solution which is a search target is supplied onto a predetermined culture substrate, and a contact angle between the culture substrate and a droplet of the culture medium solution is measured. Then, whether or not the contact angle meets a predetermined criterion (whether or not the contact angle falls within a preferable range, or whether the contact angle exceeds or falls below the criterion value) is analyzed, and it is thus possible to determine whether or not the culture medium solution is suitable for culturing the cells.

As such, it can be said that the present evaluation method can be used for searching a culture substrate and/or a culture medium solution in cell culture. Therefore, the present invention can also be said to encompass a method for searching a culture substrate and/or a culture medium solution in cell culture using the present evaluation method.

According to the present evaluation method, it is possible to evaluate suitability of a culture substrate and/or a culture medium solution used in cell culture. After that, by culturing intended cells using a culture substrate and a culture medium solution suitable for culturing the intended cells, cell culture can be carried out under conditions suitable for the cells. That is, the present invention can be said to encompass a method for culturing cells, the method including the step of evaluating suitability of a culture substrate and/or a culture medium solution used in cell culture by the foregoing evaluation method. Note that, in the method of cell culture in accordance with an aspect of the present invention, conditions suitable for culturing intended cells can be employed as appropriate as conditions of cell culture other than a combination of a culture substrate and a culture medium solution.

### EXAMPLES

The following description will discuss the present invention more specifically with reference to Examples. Note, however, that the present invention is not limited to those Examples.

[Method]

<Measurement of Contact Angle>

The contact angle was measured using a commercially available contact angle meter (Drop Master 500 available from Kyowa Interface Science Co., Ltd.) and in accordance with attached operation manuals.

Three types of commercially available dishes (respectively referred to as “dish A”, “dish B”, and “dish C”) for cell culture were used. To a surface of each of the dishes, 10 μL of pure water or a culture medium solution (PT-3001 available from Lonza Japan Ltd.) was dripped, and 60 seconds after the liquid was fixed, the contact angle was measured by a θ/2 method. Note that the contact angle was measured at room temperature (25° C.) and at atmospheric pressure.

<Cell Culture>

Human mesenchymal stem cells (PT-2501, Lonza Japan Ltd.) were used.

A cell suspension was prepared in accordance with manuals given by the cell supplier, seeded to the dishes, and incubated by static culture in a CO₂ incubator at a CO₂ concentration of 5% and at 37° C.

After culturing for a certain period of time, the culture solution was removed and washed with PBS.

After washing, the cells were observed and imaged with a microscope, and the number of adherent cells per visual field and an adhesion area of the cells were calculated. The number of adherent cells was visually counted. The adhesion area of the cells was calculated using analysis software BZ-X Analyzer of a fluorescence microscope BZ-X710 (available from Keyence Corporation).

[Results]

<Measurement of Contact Angle>

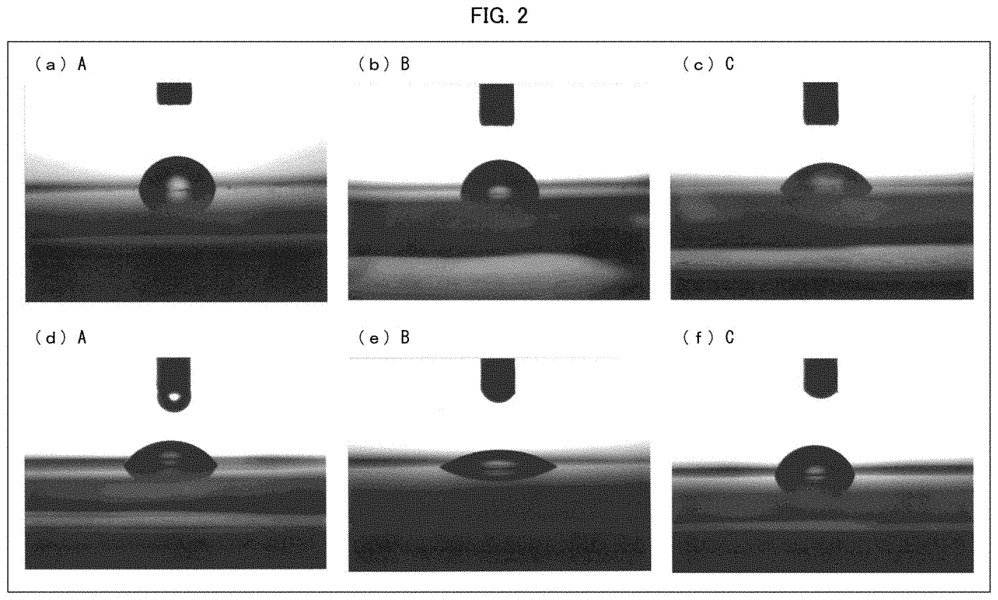

FIG. 2 is microscopic images each of which shows a droplet 60 seconds after pure water or a culture medium solution is dripped and fixed on a surface of each of dishes. (a) of FIG. 2 shows a microscopic image of a case where pure water has been dripped on the dish A. (b) of FIG. 2 shows a microscopic image of a case where pure water has been dripped on the dish B. (c) of FIG. 2 shows a microscopic image of a case where pure water has been dripped on the dish C. (d) of FIG. 2 shows a microscopic image of a case where the culture medium solution has been dripped on the dish A. (e) of FIG. 2 shows a microscopic image of a case where the culture medium solution has been dripped on the dish B. (f) of FIG. 2 shows a microscopic image of a case where the culture medium solution has been dripped on the dish C.

For (a) through (f) of FIG. 2, contact angles measured were (a) 85.8°, (b) 84.2°, (c) 59.0°, (d) 59.5°, (e) 35.0°, and (f) 77.2°. In the cases where pure water was dripped, the descending order of the contact angle was the dish A, the dish B, and the dish C. Meanwhile, in the cases where the culture medium solution was dripped, the descending order of the contact angle was the dish C, the dish A, and the dish B. As such, it has been found that the magnitude relation of contact angles measured when pure water has been dripped does not conform to the magnitude relation of contact angles measured when the culture medium solution has been dripped.

<Microscopic Observation>

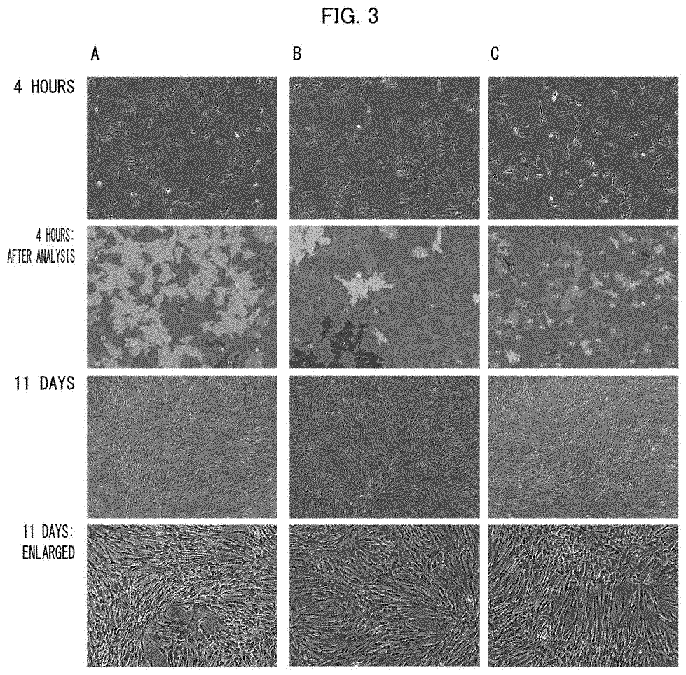

FIG. 3 shows microscopic images of cells cultured using the dishes. In FIG. 3, the microscopic images in the column A show cells cultured using the dish A; the microscopic images in the column B show cells cultured using the dish B; and the microscopic images in the column C show cells cultured using the dish C. The microscopic images in the rows labeled with “4 hours” and “11 days” in FIG. 3 are microscopic images of cells taken immediately after seeding (after 4 hours of culture) and at a time of reaching confluence (after 11 days of culture). Note that microscopic observation of cells after 4 hours of culture was carried out using a 10-times objective lens, and microscopic observation of cells after 11 days of culture was carried out using a 4-times objective lens. The microscopic images shown in the row labeled with “4 hours: After analysis” in FIG. 3 show results of image analysis of the microscopic images of the cells after 4 hours of culture using image analysis software (BZ-X Analyzer available from Keyence Corporation). The microscopic images shown in the row labeled with “11 days: Enlarged” in FIG. 3 are enlarged views of the microscopic images of cells after 11 days of culture.

When the microscopic images after 4 hours of culture are compared with each other, it can be seen that the cells spread flat on the dish A and the dish B, whereas the cells on the dish C are narrowed in shape.

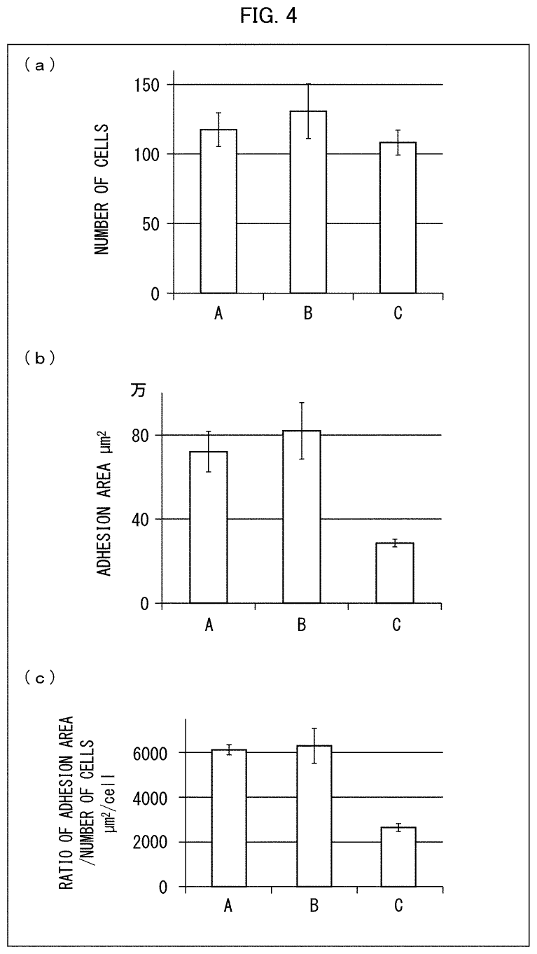

The microscopic images of the cells after 4 hours of culture were analyzed to calculate a total number of cells adhered to the dish per visual field (“number of adherent cells”) and a total area of cells adhered to the dish (“adhesion area”). The results are shown in (a) and (b) of FIG. 4. As a result, there were no clear differences in the number of adherent cells between the dishes (see (a) of FIG. 4). Meanwhile, the adhesion area of cells was clearly greater in the cases of using the dish A and the dish B, as compared with the case of using the dish C (see (b) of FIG. 4). In addition, when adhesion areas per cell are compared, it has been found that the adhesion areas per cell in the cases of using the dish A and the dish B were apparently greater, as compared with the case of using the dish C (see (c) of FIG. 4).

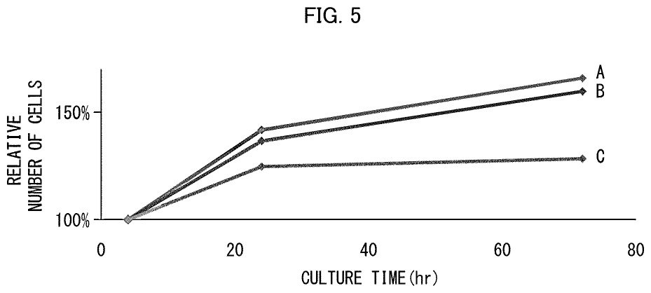

Next, the number of adherent cells per visual field was calculated from a microscopic image obtained by carrying out nuclear staining of cells using Hoechest (registered trademark) 33342 (available from DOJINDO LABORATORIES). The results are shown in FIG. 5. In FIG. 5, the number of cells after 4 hours of culture was set as 100%, and the relative numbers of cells were plotted for culture times, i.e., after 4 hours of culture, after 24 hours of culture, and after 72 hours of culture. The cell proliferation rates were then compared between the dishes. In FIG. 5, cells were grown to a similar extent in the dish A and the dish B by the culture for 72 hours. Meanwhile, in the culture using the dish C, although confluence was not achieved yet at 72 hours of culture, the growth of cells peaked at around 24 hours of cultivation.

[Main Points]

Among the results of cell culture using the dishes, better results were obtained by using the dishes A and B, as compared with the case of using the dish C (in terms of morphology of cultured cells, culture efficiency, adhesion area of cells, and the like). The culture medium solution had a higher affinity for the dish A and the dish B (the contact angle was smaller) and a lower affinity for the dish C (the contact angle was larger). Therefore, according to the Examples, it has been confirmed that the result of cell culture is improved as the affinity of the culture medium solution with the dish increases.

### INDUSTRIAL APPLICABILITY

The present invention is applicable to all industrial fields where cell culture is carried out. In particular, the present invention can be suitably utilized in industries which relate to regenerative therapy and production of substances such as pharmaceuticals using various cells.

1. A method for evaluating suitability of a culture substrate and/or a culture medium solution used in cell culture, said method comprising:
an affinity measuring step of measuring an affinity between the culture substrate and the culture medium solution; and
a suitability determining step of determining suitability of the culture substrate and/or the culture medium solution based on a result of measuring the affinity between the culture substrate and the culture medium solution which has been obtained in the affinity measuring step.

2. The method as set forth in claim 1, wherein the suitability determining step is carried out based on a suitable criterion of affinity which has been determined in advance.

3. The method as set forth in claim 1 or 2, wherein:
in the affinity measuring step, a contact angle between the culture substrate and a droplet of the culture medium solution is measured.

4. The method as set forth in claim 1, further comprising:
a criterion determining step of determining a suitable criterion of affinity between the culture substrate and the culture medium solution in advance.

5. A method for culturing cells, said method comprising the step of evaluating suitability of a culture substrate and/or a culture medium solution used in cell culture by an evaluation method recited in claim 1.

6. A method for searching a culture substrate and/or a culture medium solution used in cell culture, said method comprising the step of evaluating suitability of a culture substrate and/or a culture medium solution used in cell culture by an evaluation method recited in claim 1.
